(19) United States
(12) Patent Application Publication
(10) Pub. No.: US 2011/0136920 A1
(43) Pub. Date: Jun. 9, 2011

(54) O/W EMULSION CONTAINING A HYDROPHILIC POLYMER AND A SEMI-CRYSTALLINE POLYMER

(75) Inventor: Raluca Lorant, Thiais (FR)

(73) Assignee: L'OREAL, Paris (FR)

(21) Appl. No.: 12/304,009

(22) PCT Filed: May 24, 2007
(86) PCT No.: PCT/EP2007/055036

§ 371 Date: Mar. 11, 2009

Related U.S. Application Data
(60) Provisional application No. 60/813,716, filed on Jun. 15, 2006.

(30) Foreign Application Priority Data
Jun. 9, 2006 (FR) 0652505

(51) Int. Cl. A61K 8/81; A61Q 19/00; A61Q 1/00

(52) U.S. Cl. 514/772.4

(57) ABSTRACT

The invention relates to a composition for topical application, in the form of an O/W emulsion containing at least one crosslinked 2-acrylamido-2-methylpropane-sulfonic acid polymer comprising at least one hydrophobic unit, and at least one semi-crystalline polymer. The composition obtained has very good stability, and also good cosmetic properties. The present invention also relates to the uses of the said composition in cosmetics and dermatology, especially for caring for, protecting and/or making up facial or bodily skin, and/or the lips, and/or for skincare, and more especially for treating dry and/or sensitive skin and for treating the signs of ageing of the skin.

The invention relates to a composition for topical application in the form of an O/W emulsion containing a particular hydrophilic polymer and a semi-crystalline polymer, and to the use of the said composition in cosmetics or dermatology, and in particular for caring for, protecting and/or making up bodily or facial skin, or for haircare.

In the cosmetics field, the addition of waxes or of similar fatty substances to skincare compositions makes it possible to obtain products that are particularly suitable for treating dry skin and aged skin. Waxes especially contribute towards nourishing the skin and smoothing out wrinkles and fine lines. They may be introduced either into W/O emulsions or into O/W emulsions. Generally, in the cosmetics field, O/W emulsions are preferred to W/O emulsions on account of their cosmetic qualities that are judged to be superior since they are often lighter and fresher, and also on account of their greater flexibility of formulation. However, the presence of waxes in standard O/W emulsions results in impairment in the stability of these emulsions, whereas their introduction into W/O emulsions poses no problems. Moreover, the cosmetic properties of O/W emulsions containing waxes are often judged to be insufficient; they are especially often found to have an excessively thick consistency, to be difficult to spread and to produce a greasy film after application to the skin.

Emulsifying surfactants are usually used to emulsify and stabilize compositions that are rich in waxy and pasty fatty substances and in particular rich in waxes. However, these surfactants are known to be potentially irritant, especially to sensitive skin, causing discomfort such as itching, dehydration and coarse skin. In addition, co-emulsifying agents are often added thereto, and substances used as co-emulsifying agents, such as fatty alcohols, may reinforce the sensory discomfort, such as the greasy and tacky feel and the coarse effect, and give compositions that are difficult to spread. There are currently no products on the market in the form of an O/W emulsion containing waxes and having low contents of emulsifiers, i.e. less than 2%.

Furthermore, this type of product rich in waxes or, in general, rich in fatty substances, and containing surfactants, has thick and greasy textures, which lack freshness and sensory pleasantness.

There is thus still a need for O/W emulsions that nourish the skin, and thus that contain skin-nourishing fatty substances such as waxes, while at the same time having textures that are easy to apply and pleasant to use, and that are neither greasy, tacky nor potentially irritant. This type of product is particularly advantageous for treating dry skin, and also for fragile and sensitive skin due to the fact that it may be free of surfactants, to which sensitive skin is particularly reactive. Furthermore, freshness effects and easy application are particularly advantageous for soothing dry and reactive skin.

The Applicant has discovered, unexpectedly, that the use of a combination of particular hydrophilic polymers and of particular lipophilic polymers makes it possible to solve the underlying problem of the invention and to obtain a stable O/W emulsion that has good cosmetic properties, even in the presence of fatty substances such as waxes, and that may be advantageously free of emulsifying surfactant conventionally used in O/W emulsions or contain only a very small amount thereof, and thus be well tolerated by any skin type.

These compositions may contain fatty substances, including waxy and pasty fatty substances, while at the same time having good stability and surprising sensory pleasantness, i.e. a feel that is both rich and fresh. These surprising properties are demonstrated in various tests presented later.

In addition, these surprising properties are valid even for very thick compositions. Specifically, even when they are rich in thickeners and waxy and/or pasty fatty substances and when they have a high viscosity with the consistency of a thick balm, these compositions provide a freshness effect that is experienced immediately on application, and, in addition, they do not leave any greasy or tacky residue on the skin after penetration. Thus, even when their viscosity is high, these emulsions “melt” on contact with the skin, allowing very easy application. These sensory qualities combining richness in fatty substances, freshness and easy application are very surprising, all the more so since their consistency is thick, and they participate both towards the pleasantness of the compositions according to the invention and to their efficacy in treating the skin, especially in treating dry and/or sensitive skin.

Another surprising characteristic of the products according to the invention is that of combining the qualities of “waxy” products (very specific feel) with a sensation of freshness. Specifically, the known products that have a “waxy” feel never have a freshness effect.

One subject of the invention is thus a composition for topical application, in the form of an O/W emulsion comprising an oily phase dispersed in an aqueous phase, characterized in that it contains at least one crosslinked polymer of 2-acrylamido-2-methylpropane-sulfonic acid comprising at least one hydrophobic unit, and at least one semi-crystalline polymer.

Since the composition according to the invention is intended for topical application to the skin or the integuments, it contains a physiologically acceptable medium. The term “physiologically acceptable medium” means a medium that is compatible with the skin, the lips, the nails, the scalp and/or the hair.

The emulsion obtained according to the invention shows good stability over time, even at a temperature above room temperature (for example 45° C.). The term “stable emulsion” means an emulsion which, after storage for 24 hours at any temperature between 4° C. and 50° C., shows no macroscopic change in colour, odour or viscosity or any variation in pH.

The composition of the invention is in the form of a more or less fluid cream, i.e. a soft product, as opposed to a solid product such as a stick. This cream may have a texture ranging from fluid to thick. Thus, it may be very fluid like a milk or very thick like a balm. This composition may have, for example, a viscosity at room temperature (25° C.) that varies over a very wide range, for example a viscosity ranging from about 4 to 500 poises (0.4 to 50 Pa·s) and preferably from about 10 to 450 poises (1 to 45 Pa·s), this viscosity being measured with a Rheomat 180 viscometer at 25° C., with spindles adapted to the viscosity, especially No. 3, 4 or 5 spindles.

Crosslinked 2-acrylamido-2-methylpropanesulfonic Acid Polymers

The hydrophilic polymers used in the composition of the invention are crosslinked 2-acrylamido-2-methylpropane-sulfonic acid (AMPS) polymers comprising at least one hydrophobic group; they are hydrophobic modified AMPS crosslinked polymers. They are copolymers based on at least one AMPS hydrophilic monomer and on at least one hydrophobic monomer. These polymers are water-soluble or water-dispersible, in neutralized or partially neutralized form. They are preferably totally neutralized. Since the polymers are water-soluble or water-dispersible, they are generally introduced into the aqueous phase of the emulsion.

The term “water-soluble or water-dispersible polymers” means polymers which, when introduced into water to a concentration equal to 1% by weight, give a macroscopically homogeneous solution whose light transmittance, at a wavelength equal to 500 nm, through a sample 1 cm thick, is at least 10%.

The term “crosslinked polymer” means a polymer that has reacted with crosslinking agents and thus comprises at least one crosslinking unit.

The polymers of the invention generally have a weight-average molecular mass ranging from 10 000 to 10 000 000, more preferentially from 100 000 to 8 000 000 and even more preferentially from 100 000 to 7 000 000.

Hydrophobic modified AMPS crosslinked polymers that may especially be used include those comprising:
80 mol % to 99 mol % and preferably from 85 mol % to 99 mol % of 2-acrylamido-2-methylpropanesulfonic acid (AMPS) units of formula (I):

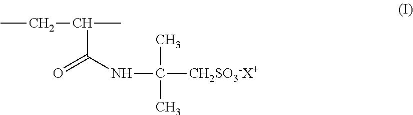

in which X⁺ is a proton, an alkali metal cation, an alkaline-earth metal cation, an ammonium ion or an organic cation; and
1 mol % to 20 mol % and preferably from 1 mol % to 15 mol % of hydrophobic units of formula (II) below:

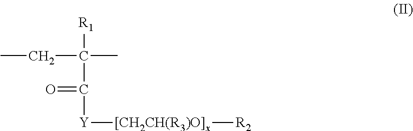

in which R₁ and R₃, which may be identical or different, denote a hydrogen atom or a substantially linear or branched C₁-C₆ alkyl radical (preferably methyl); Y denotes O or NH; R₂ denotes a hydrocarbon-based radical containing from 6 to 50 carbon atoms, more preferably from 6 to 22 carbon atoms, even more preferably from 6 to 18 carbon atoms and more particularly from 12 to 18 carbon atoms; and x denotes a number of moles of alkylene oxide and ranges from 0 to 100 and better still from 3 to 100.

The radical R₂ is preferably chosen from substantially linear (for example n-hexyl, n-octyl, n-decyl, n-hexadecyl, n-dodecyl or lauryl, n-octadecyl, stearyl or behenyl), branched or cyclic C₆-C₁₈ alkyl radicals (for example cyclododecane (C₁₂) or adamantane (C₁₀)); C₆-C₁₈ perfluoroalkyl radicals (for example the group of formula —(CH₂)₂—(CF₂)₉—CF₃); the cholesteryl radical (C₂₇) or a cholesterol ester residue, for instance the cholesteryl oxyhexanoate group; aromatic polycyclic groups, for instance naphthalene or pyrene. Among these radicals, linear alkyl radicals, and more particularly stearyl and behenyl radicals, and mixtures thereof, are more particularly preferred.

According to one particularly preferred form of the invention, the monomer of formula (II) comprises at least one alkylene oxide unit (x≧1) and preferably several alkylene oxide units (x>1) constituting a polyoxyalkylenated chain. The polyoxyalkylenated chain preferably consists of ethylene oxide units and/or propylene oxide units and even more particularly consists of ethylene oxide units. The number of oxyalkylene units (or number of moles of alkylene oxide) generally ranges from 3 to 100, more preferentially from 3 to 50 and even more preferentially from 7 to 25.

Preferably, the polymer contains as hydrophobic unit of formula (II), a unit of formula (III):

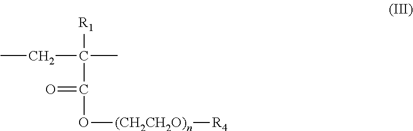

in which n denotes an integer ranging from 3 to 100, preferably from 3 to 50 and more preferentially from 7 to 25; R₁ is hydrogen or a methyl radical and R₄ denotes a linear or branched alkyl radical containing from 6 to 22 carbon atoms, preferably from 10 to 22 carbon atoms and better still from 14 to 22 carbon atoms.

The hydrophobic modified AMPS polymers that may be used in the composition in accordance with the invention may be obtained according to the standard free-radical polymerization processes in the presence of one or more initiators, for instance azobisisobutyronitrile (AIBN), azobisdimethylvaleronitrile, 2,2-azobis[2-amidino-propane]hydrochloride (ABAH), organic peroxides such as dilauryl peroxide, benzoyl peroxide, tert-butyl hydroperoxide, etc., mineral peroxide compounds such as potassium persulfate or ammonium persulfate, or H₂O₂ optionally in the presence of reducing agents.

These hydrophobic modified polymers may especially be obtained by free-radical polymerization in tert-butanol medium, from which they precipitate. By using polymerization by precipitation in tert-butanol, it is possible to obtain a size distribution of the polymer particles that is particularly favourable for its uses.

The reaction may be performed at a temperature of between 0 and 150° C. and preferably between 10 and 100° C., either at atmospheric pressure or under reduced pressure. It may also be performed under an inert atmosphere and preferably under nitrogen.

The hydrophobic modified crosslinked AMPS polymers used in the composition of the invention are preferably in partially or totally neutralized form. The term “partially neutralized polymers” means polymers that are at least 90% neutralized. According to one preferred embodiment of the invention, they are totally neutralized.

Preferentially, the partial or total neutralization of the AMPS polymers used in accordance with the invention is performed using a mineral base (sodium hydroxide, potassium hydroxide or aqueous ammonia) or an organic base such as monoethanolamine, diethanolamine, triethanolamine, an aminomethylpropanediol, N-methyl-glutamine, or basic amino acids, for instance arginine and lysine, and mixtures of these compounds.

The polymers used according to the invention are crosslinked. The crosslinking agents may be chosen from the polyolefinically unsaturated compounds commonly used for crosslinking polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl(meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylene-bisacrylamide, allyl methacrylate and trimethylol-propane triacrylate (TMPTA). The degree of crosslinking generally ranges from 0.01 mol % to 10 mol % and more particularly from 0.2 mol % to 2 mol % relative to the polymer.

Among these polymers, mention may be made of:
the crosslinked AMPS copolymers described generally in document EP-A-1 069 142;
crosslinked or non-crosslinked and neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl(meth)acrylate units, relative to the polymer, such as those described in patent application EP-A-750 899;
crosslinked copolymers of partially or totally neutralized AMPS and of n-dodecylmethacrylamide, such as those described in the Morishima articles cited above.

As polymers that are more particularly suitable, mention may be made of those obtained by polymerization of 2-acrylamido-2-methylpropanesulfonic acid (AMPS) or a sodium or ammonium salt thereof, with a methacrylic or acrylic acid ester, preferably an ester of methacrylic acid and of an oxyethylenated alcohol of formula (IV):

—CH═C(CH₃)—CO—O(CH₂CH₂O)n-R (IV)

in which n ranges from 7 to 25, and is preferably equal to 8 or 25, and R is an alkyl residue containing from 10 to 22 carbon atoms.

Polymers of this type that may be mentioned more especially include the product sold under the name Aristoflex HMS by the company Clariant, which is an AMPS/ethoxylated (25 EO) stearyl methacrylate crosslinked copolymer, for which in formula (IV) n is 25 and R is C₁₆-C₁₈, or the product sold under the name Aristoflex HMB by the company Clariant, which is an AMPS/ethoxylated (25 EO) behenyl methacrylate crosslinked copolymer, for which in formula (IV) n is 25 and R is C22. A mixture of these polymers may also be used.

The amount of AMPS polymer containing a hydrophobic unit in the composition of the invention may range, for example, as active material, from 0.1% to 5% by weight, preferably from 0.2% to 5% by weight and better still from 0.2% to 3% by weight relative to the total weight of the composition.

### Semi-Crystalline Polymers

The composition according to the invention contains at least one semi-crystalline polymer, preferably derived from acrylic or methacrylic acid. The term “semi-crystalline polymer” means polymers comprising a crystallizable portion, a crystallizable side chain or a crystallizable block in the skeleton, and an amorphous portion in the skeleton and having a first-order reversible phase-change temperature, in particular of melting (solid-liquid transition). When the crystallizable portion is in the form of a crystallizable block of the polymer skeleton, the amorphous portion of the polymer is in the form of an amorphous block; in this case, the semi-crystalline polymer is a block copolymer, for example, of the diblock, triblock or multiblock type.

Advantageously, the semi-crystalline polymer(s) of the composition of the invention have a number-average molecular mass M̅n greater than or equal to 2000, for example ranging from 2000 to 800 000, preferably from 3000 to 500 000, for example from 4000 to 150 000 and better still from 4000 to 99 000.

In the composition according to the invention, the semi-crystalline polymers are advantageously soluble in the oily phase to at least 1% by weight, at a temperature above their melting point. Besides the crystallizable chains or blocks, the polymer blocks are amorphous. For the purposes of the invention, the term “crystallizable chain or block” means a chain or block which, if it were alone, would pass reversibly from the amorphous state to the crystalline state, depending on whether it is above or below its melting point. For the purposes of the invention, a chain is a group of atoms, which is pendent or lateral relative to the polymer backbone. A block is a group of atoms belonging to the backbone, this group constituting one of the repeating units of the polymer.

Preferably, the polymer backbone of the semi-crystalline polymers is soluble in the oily phase.

Preferably, the semi-crystalline polymers used in the composition of the invention have a melting point, m.p., of less than 70° C. (25° C.≦m.p.<70° C.), this temperature being at least equal to the temperature of the keratin material that is to receive the composition according to the invention, especially the skin. The melting point may be measured especially by any known method and in particular with a differential scanning calorimeter (DSC).

Preferably, the crystallizable blocks or chains of the semi-crystalline polymers represent at least 30% and better still at least 40% of the total weight of each polymer. The semi-crystalline polymers containing crystallizable blocks used according to the invention are block or multiblock polymers. They may be obtained by polymerization of monomers containing reactive double bonds (or ethylenic bonds) or by polycondensation. When the polymers of the invention are polymers containing crystallizable side chains, they are advantageously in random or statistical form.

The semi-crystalline polymers of the invention are of synthetic origin. In addition, they comprise no polysaccharide backbones.

The semi-crystalline polymers that may be used in the invention are preferably chosen from polymers (homopolymers or copolymers) bearing at least one crystallizable side chain, and polymers (homopolymers or copolymers) bearing in the backbone at least one crystallizable block, such as those described in document U.S. Pat. No. 5,156,911. The crystallizable side chains or blocks are hydrophobic.

According to one preferred embodiment of the invention, the semi-crystalline polymers are especially chosen from homopolymers and copolymers resulting from the polymerization of at least one monomer containing (a) crystallizable side chain(s) chosen from alkyl chains containing at least 11 carbon atoms and not more than carbon atoms and better still not more than 24 carbon atoms. They are especially alkyl chains containing at least 12 carbon atoms, and are preferably alkyl chains containing from 14 to 24 carbon atoms (C₁₄-C₂₄). They may be hydrocarbon-based alkyl chains (carbon and hydrogen atoms) or fluoro or perfluoro alkyl chains (carbon atoms, fluorine atoms and possibly hydrogen atoms). When they are fluoro or perfluoro alkyl chains, they contain at least 11 carbon atoms, at least 6 carbon atoms of which are fluorinated.

For the purposes of the invention, the term “alkyl” means a saturated group (containing no unsaturation).

According to one particular embodiment of the invention, the semi-crystalline polymer is chosen from polymers derived from at least one monomer with a crystallizable chain chosen from C₁₄-C₂₄ alkyl (meth)acrylates, C₁₁-C₁₅ perfluoroalkyl(meth)acrylates, C₁₄-C₂₄ N-alkyl(meth)acrylamides with or without fluorine atoms, vinyl esters containing C₁₄-C₂₄ alkyl or perfluoroalkyl chains, vinyl ethers containing C₁₄-C₂₄ alkyl or perfluoroalkyl chains, C₁₄-C₂₄ α-olefins, para-alkylstyrenes with a C₁₄-C₂₄ alkyl group, and copolymers of these monomers obtained by copolymerization of these monomers with a hydrophilic monomer, which is preferably other than methacrylic acid, for instance N-vinylpyrrolidone, hydroxyethyl acrylate, hydroxyethyl methacrylate or acrylic acid. Such copolymers may be, for example, copolymers of (C₁₄-C₂₄)alkyl acrylate, of (C₁₄-C₂₄) alkyl methacrylate, of (C₁₄-C₂₄) alkylacrylamide or of (C₁₄-C₂₄)alkylmethacrylamide with N-vinylpyrrolidone, hydroxyethyl acrylate, hydroxyethyl methacrylate or acrylic acid, or mixtures thereof.

The semi-crystalline polymers of the composition of the invention may be non-crosslinked or partially crosslinked, provided that the degree of crosslinking does not hinder their dissolution or dispersion in the oily phase by heating above their melting point. It may then be a case of chemical crosslinking, by reaction with a multifunctional monomer during the polymerization. It may also be a case of physical crosslinking, which may then be due either to the establishment of bonds of hydrogen or dipolar type between groups borne by the polymer, for instance dipolar interactions between carboxylate ionomers, these interactions being in small amount and borne by the polymer backbone; or a phase separation between the crystallizable blocks and the amorphous blocks borne by the polymer.

Preferably, the semi-crystalline polymers of the composition according to the invention are non-crosslinked.

According to one particular embodiment of the invention, the polymer is a homopolymer resulting from the polymerization of a monomer with crystallizable chains chosen from C₁₄-C₂₂ alkyl acrylates and C₁₄-C₂₂ alkyl methacrylates. Mention may be made especially of those sold under the name Intelimer® by the company Landec, described in the brochure “Intelimer® polymers”, Landec IP22 (Rev. 4-97). These polymers are in solid form at room temperature. They bear crystallizable side chains and correspond to saturated C₁₄-C₂₄ alkyl acrylate or methacrylate homopolymers. Mention may be made more particularly of the stearyl acrylate homopolymer (Intelimer IPA-13.1) (INCI name: Poly C10-30 alkyl acrylate) and the behenyl acrylate homopolymer (Intelimer IPA-13.6) (INCI name: Poly C10-30 alkyl acrylate).

It is also possible to use copolymers of C₁₄-C₂₂ alkyl acrylates or of C₁₄-C₂₂ alkyl methacrylates with, especially, acrylic acid. Copolymers that may be mentioned include copolymers obtained by copolymerization of behenyl acrylate and of acrylic acid, or copolymers obtained by copolymerization of stearyl acrylate and of acrylic acid.

The amount of semi-crystalline polymer(s) in the composition of the invention may range, for example, from 0.1% to 5% by weight, preferably from 0.5% to 5% by weight and better still from 1% to 3% by weight relative to the total weight of the composition.

### Oily Phase

The oily phase of the composition according to the invention contains at least one fatty substance of animal, plant, mineral or synthetic origin, chosen from volatile or non-volatile oils that are liquid at room temperature (20-25° C.), waxes, gums and other lipophilic fatty substances, and mixtures thereof. These fatty substances are physiologically acceptable.

The amount of oily phase preferably represents from 2% to 40% by weight and better still from 5% to 30% by weight relative to the total weight of the composition.

According to one preferred embodiment of the invention, the oily phase contains at least one oil. The term “oil” means a fatty substance that is liquid at room temperature (25° C.).

As oils that may be used in the composition of the invention, examples that may be mentioned include:
hydrocarbon-based oils of animal origin, such as perhydrosqualene (or squalane);
hydrocarbon-based oils of plant origin, such as liquid triglycerides of fatty acids containing from 4 to 10 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, coriander oil, castor oil, avocado oil, caprylic/capric acid triglycerides, for instance those sold by the company Stearineries Dubois or those sold under the names Miglyol 810, 812 and 818 by the company Dynamit Nobel, jojoba oil, shea butter oil and liquid fractions of shea butter, and perhydrosqualene of plant origin;
synthetic esters and ethers, especially of fatty acids, for instance the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, for instance purcellin oil, isononyl isononanoate, isopropyl myristate, 2-ethylhexyl palmitate (or octyl palmitate), 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate; lipophilic amino acid derivatives, such as isopropyl lauroyl sarcosinate (INCI name: Isopropyl lauroyl sarcosinate) sold under the name Eldew SL 205 by the company Ajinomoto;
linear or branched hydrocarbons of mineral or synthetic origin, such as mineral oils (mixture of hydrocarbon-based oils derived from petroleum; INCI name: Mineral oil), volatile or non-volatile liquid paraffins, and derivatives thereof, petroleum jelly, polydecenes, isohexadecane, isododecane, and hydrogenated isoparaffin such as Parleam® oil, sold by the company NOF Corporation (INCI name: Hydrogenated polyisobutene);
fatty alcohols containing from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol;
partially hydrocarbon-based and/or silicone-based fluoro oils, for instance those described in document JP-A-2 295 912;
silicone oils, for instance volatile or non-volatile polymethylsiloxanes (PDMSs) containing a linear or cyclic silicone chain, which are liquid or pasty at room temperature, especially volatile silicone oils which are either linear or cyclic, for instance cyclopolydimethylsiloxanes (cyclomethicones) such as cyclopentasiloxane and cyclohexasiloxane; polydimethyl-siloxanes comprising alkyl, alkoxy or phenyl groups, which are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyl-dimethicones, diphenylmethyldiphenyltrisiloxanes, 2-phenylethyltrimethylsiloxysilicates and polymethyl-phenylsiloxanes;
mixtures thereof.

According to one particular embodiment of the invention, the oily phase of the composition of the invention contains at least one wax or one pasty fatty substance. The amount of wax(es) and of pasty fatty substance(s) in the composition of the invention may range, for example, from 0.05% to 10% by weight, better still from 0.1% to 10% by weight and even better still from 0.5% to 5% by weight relative to the total weight of the composition.

The term “waxes” means fatty substances that are solid at room temperature (25° C.) and that have a melting point of greater than 30° C. (measured by DSC), and better still greater than 40° C.

For the purposes of the patent application, the waxes are those generally used in cosmetics and dermatology; they may be hydrocarbon-based waxes, silicone waxes and/or fluoro waxes, optionally comprising ester or hydroxyl functions. It is possible, for example, to use mineral waxes; waxes of animal origin; waxes of plant origin; hydrogenated oils that are solid at 25° C., fatty esters and glycerides that are solid at 25° C., synthetic waxes and silicone waxes, and mixtures thereof.

As examples of waxes that may be used in the composition of the invention, mention may be made of microcrystalline waxes; paraffin waxes; lignite waxes; ceresin; ozokerite; montan wax; carnauba wax; jojoba wax; olive wax (hydrogenated fatty alcohol ester of olive oil); beeswax; lanolin and its derivatives; candelilla wax, ouricury wax; Japan wax; cocoa butter; palm oil in paste form at 20° C.; cork fibre wax or sugarcane wax; hydrogenated oils that are solid at 25° C. such as palm oil and hydrogenated palm kernel oil, and hydrogenated castor oil; fatty esters such as hydrogenated castor oil esters sold under the name Risocast by the company Kokyu Alcohol Kogyo; glycerides that are solid at 25° C.; polyethylene or polymethylene waxes and the waxes obtained by Fischer-Tropsch synthesis; silicone waxes, and mixtures thereof.

Pasty fatty substances that may be mentioned include petrolatum and petroleum jelly; and also butters such as shea butter, cocoa butter, shorea butter, muru muru butter and cupuacu butter, and mixtures thereof.

According to one particularly preferred embodiment of the invention, the composition according to the invention contains at least one wax or one pasty fatty substance chosen from carnauba wax, beeswax, polyethylene waxes, polymethylene waxes, shea butter or other butters of plant origin, petrolatum and petroleum jelly, and mixtures thereof.

According to one particularly preferred embodiment, the composition of the invention contains at least one oil and at least one wax and/or at least one pasty fatty substance.

The other fatty substances that may be present in the oily phase are, for example, fatty acids containing from 8 to 30 carbon atoms, for instance stearic acid, lauric acid or palmitic acid; fatty alcohols comprising from 8 to 30 carbon atoms, for instance stearyl alcohol, cetyl alcohol and mixtures thereof (cetearyl alcohol); gums such as silicone gums (dimethiconol); silicone resins such as trifluoromethyl-C₁₋₄ alkyldimethicone and trifluoropropyl dimethicone, and silicone elastomers, for instance the products sold under the name “KSG” by the company Shin-Etsu, under the name “Trefil” by the company Dow Corning or under the name “Gransil” by the company Grant Industries; and mixtures thereof.

### Aqueous Phase

The aqueous phase of the composition of the invention may range from 60% to 98% by weight and preferably from 70% to 95% by weight relative to the total weight of the composition. It contains at least water. Besides water, it may contain one or more water-miscible or at least partially water-miscible compounds, for instance polyols; C₂ to C₈ lower monoalcohols, such as ethanol and isopropanol; and C₃ to C₄ ketones that are liquid at room temperature. The term “room temperature” should be understood as meaning a temperature of about 25° C. at normal atmospheric pressure (760 mmHg).

The term “polyol” should be understood as meaning any organic molecule comprising at least two free hydroxyl groups. Examples of polyols that may be mentioned include glycerol, glycols, for instance butylene glycol, propylene glycol, isoprene glycol, dipropylene glycol, hexylene glycol, pentylene glycol and polyethylene glycols, for instance PEG-8, sorbitol and sugars, for instance glucose.

The solvent(s) may be present in an amount ranging from 0.1% to 30% by weight and better still from 0.5% to 15% by weight relative to the total weight of the composition.

### Adjuvants

In a known manner, the composition of the invention may also contain adjuvants that are common in cosmetics and/or dermatology, such as active agents, preserving agents, antioxidants, complexing agents, pH regulators (acidic or basic), fragrances, fillers, bactericides, odour absorbers, dyestuffs (pigments and dyes), surfactants, polymers or lipid vesicles. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

As polymers that may be used in the composition according to the invention, other than the polymers used according to the invention, mention may be made especially of hydrophilic polymers such as modified or unmodified carboxyvinyl polymers, such as the products sold under the names Carbopol (INCI name: Carbomer) and Pemulen (INCI name: Acrylates/C10-30 alkyl acrylate crosspolymer) by the company Goodrich, or such as the crosslinked sodium polyacrylate sold under the name Cosmedia SP by the company Cognis (INCI name: Sodium polyacrylate); polyacrylamides; homopolymers derived from 2-acrylamido-2-methylpropanesulfonic acid, such as the product sold by Clariant under the name Hostacerin AMPS (INCI name: ammonium polyacryldimethyltaurate); crosslinked anionic copolymers of acrylamide and of AMPS, in the form of an emulsion, such as those sold under the name Sepigel 305 (CTFA name: Polyacrylamide C13-14 Isoparaffin/Laureth-7) and under the name Simulgel 600 (CTFA name: Acrylamide/Sodium acryloyldimethyltaurate copolymer/-Isohexadecane/Polysorbate 80) by the company SEPPIC; acrylate/acrylonitrile copolymers such as HYPAN SS201 sold by the company Kingston; synthetic neutral polymers such as poly-N-vinylpyrrolidone; poly-saccharides, for instance guar gum, xanthan gum and cellulose derivatives. The amount of these polymers may range, for example, from 0.05% to 5% by weight and better still from 0.1% to 3% by weight relative to the total weight of the composition.

The composition according to the invention may contain one or more fillers. Examples of fillers that may be mentioned include the pigments such as titanium oxide, zinc oxide or iron oxide and organic pigments; kaolin; silica; talc; boron nitride; organic spherical powders, fibres; and mixtures thereof. Examples of organic spherical powders that may be mentioned include polyamide powders and especially Nylon® powders such as Nylon-1 or Polyamide 12, sold under the name Orgasol by the company Atochem; polyethylene powders; Teflon®; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; polymethyl methacrylate microspheres, sold under the name Microsphere M-100 by the company Matsumoto or under the name Covabead LH85 by the company Wackherr; powders of natural organic materials such as starch powders, especially of maize starch, wheat starch or rice starch, which may or may not be crosslinked, such as the starch powders crosslinked with octenyl succinate anhydride, sold under the name Dry-Flo by the company National Starch; and mixtures thereof. These fillers may be present in amounts ranging from 0 to 20% by weight and preferably from 0.5% to 10% by weight relative to the total weight of the composition.

The composition may contain one or more active agents. Active agents that may especially be used include vitamins and derivatives thereof, especially esters thereof, such as retinol (vitamin A) and its esters (for example retinyl palmitate), ascorbic acid (vitamin C) and its esters (for example magnesium ascorbyl phosphate and ascorbyl glucoside), tocopherol (vitamin E) and its esters (for example tocopheryl acetate), vitamin B3 or B10 (niacinamide and derivatives), alone or as a mixture; keratolytic and/or desquamating agents such as salicylic acid and its derivatives, α-hydroxy acids, for instance lactic acid, citric acid and glycolic acid; anti-inflammatory agents; calmatives such as allantoin and bisabolol; depigmenting agents; tensioning agents such as synthetic polymers, plant proteins, polysaccharides of plant origin, starches, wax dispersions, mixed silicates and colloidal particles of mineral fillers; matting agents; or anti-wrinkle agents, and mixtures thereof.

The composition may contain one or more sunscreens. Examples of sunscreens that may be mentioned include organic UV-screening agents such as:
Salicylic derivatives, and especially ethylhexyl salicylate (or ethylhexyl salicylate) sold under the trade name Neo Heliopan OS by Haarmann and Reimer;
Dibenzoylmethane derivatives, and especially butyl methoxydibenzoylmethane sold in particular under the trade name Parsol 1789 by Hoffmann LaRoche;
Cinnamic derivatives, and especially ethylhexyl methoxycinnamate sold in particular under the trade name Parsol MCX by Hoffmann LaRoche;
β,β′-Diphenylacrylate derivatives, and especially octocrylene (2-ethylhexyl α-cyano-β,β-diphenyl-acrylate), sold in particular under the trade name Uvinul N539 by BASF;
Phenylbenzimidazolesulfonic acid;
Benzylidenecamphor derivatives, and especially terephthalylidenedicamphorsulfonic acid manufactured under the name Mexoryl SX by Chimex, and 4-methylbenzylidenecamphor sold under the trade name Eusolex 6300 by Merck;
Benzophenone derivatives, and especially benzophenone-3 or Oxybenzone sold under the trade name Uvinul M40 by BASF; benzophenone-4 sold under the trade name Uvinul MS40 by BASF; benzophenone-5;
Phenylbenzimidazole derivatives, and especially Benzimidazilate sold under the trade name Neo Heliopan AP by Haarmann and Reimer;
Triazine derivatives, and especially anisotriazine sold under the trade name Tinosorb S by Ciba Geigy; ethylhexyltriazone sold in particular under the trade name Uvinul T150 by BASF; and diethylhexylbutamidotriazone sold under the trade name Uvasorb HEB by Sigma 3V;
Methylenebis(benzotriazolyl)tetramethylbutylphenol;
Phenylbenzotriazole derivatives, and especially drometrizole trisiloxane sold under the trade name Silatrizole by Rhodia Chimie; or methylenebis-(benzotriazolyl)tetramethylbutylphenol, sold in solid form under the trade name MIXXIM BB/100 by Fairmount Chemical, or in micronized form as an aqueous dispersion under the trade name Tinosorb M by Ciba Specialty Chemicals;
and mixtures thereof.

The sunscreens may also be chosen from physical sunblocks. As examples of physical sunblocks that may be added to the composition of the invention, mention may be made of pigments and nanopigments of coated or uncoated metal oxides, especially titanium oxide, iron oxide, zirconium oxide, zinc oxide or cerium oxide, and mixtures thereof, these oxides possibly being in the form of optionally coated microparticles or nanoparticles (nanopigments).

The amount of active agents depends on the desired aim. The active agent(s) may be present, for example, in a concentration ranging from 0.01% to 20%, preferably from 0.05% to 10% and better still from 0.1% to 5% by weight relative to the total weight of the composition.

Needless to say, a person skilled in the art will take care to select the optional compound(s) to be added to the composition according to the invention and the amounts thereof, such that the advantageous properties intrinsically associated with the composition in accordance with the invention are not, or are not substantially, adversely affected by the envisaged addition.

According to one particular embodiment of the invention, the composition according to the invention may be substantially free of emulsifying surfactant usually used in O/W emulsions. The term “substantially free of surfactant” refers to a composition containing less than 0.5% by weight and preferably less than 0.3% by weight of emulsifying surfactant relative to the total weight of the composition, or even being totally free of any emulsifier.

However, the composition according to the invention may also contain emulsifying surfactants chosen from hydrophilic emulsifiers that are gentle to the skin, and especially sugar-based emulsifiers, such as fatty acid esters of sugars, and fatty alkyl ethers of sugars, and mixtures thereof, i.e. esters or mixtures of esters of a C₈-C₂₂ fatty acid and of sucrose, maltose, glucose, methylglucose or fructose, and ethers or mixtures of the ethers of a C₈-C₂₂ fatty alcohol and of glucose, maltose, sucrose, methylglucose or fructose.

Examples of sugar-based emulsifiers that may be mentioned include sucrose mono-di-palmito-stearate (INCI name: Sucrose stearate) such as the product sold under the name Tegosoft PSE 141 G by the company Goldschmidt, sucrose monostearate, sucrose distearate and sucrose tristearate, and mixtures thereof, such as the products sold by the company Croda under the name Crodesta F50, F70, F110 or F160, methylglucose polyglyceryl-3 distearate, sold by the company Goldschmidt under the name Tegocare 450, alkylpolyglucosides (APG) such as decyl glucoside and lauryl glucoside sold, for example, by the company Cognis under the respective names Plantaren 2000 and Plantaren 1200, cetostearyl glucoside optionally as a mixture with cetostearyl alcohol, sold under the name Montanov 68 by the company SEPPIC, under the name Tegocare CG90 by the company Goldschmidt and under the name Emulgade KE3302 by the company Henkel, and also arachidyl glucoside, for example in the form of a mixture of arachidyl alcohol, behenyl alcohol and arachidyl glucoside, sold under the name Montanov 202 by the company SEPPIC.

A co-emulsifier may advantageously be added, such as stearic acid that is non-neutralized or partially neutralized with a base such as sodium hydroxide or triethanolamine, such that the pH is between 5 and 7.

If the composition contains an emulsifier, the amount of emulsifier and of co-emulsifier is preferably less than 3%, and ranges, for example, from 0.1% to 3% by weight and better still from 0.2% to 2% by weight relative to the total weight of the composition.

As indicated above, the composition according to the invention may especially be in the form of a more or less fluid or thick cream and may especially constitute a cosmetic or dermatological composition. It then finds its application in a large number of treatments, especially of cosmetic treatments of the skin, including the scalp, and of mucous membranes, in particular for caring for, protecting, making up with addition of dyes or pigments, or removing makeup from bodily or facial skin or the lips.

Thus, a subject of the present invention is in particular the cosmetic use of a cosmetic composition as defined above, for caring for, protecting and/or making up the skin and/or the lips.

The present invention more particularly relates to the use of the composition as described, as a care product for treating wrinkles and the signs of ageing of the skin.

The composition according to the invention is, for example, suitable for reducing wrinkles and fine lines on the skin and also for moisturizing the skin.

A subject of the present invention is thus also the cosmetic use of a cosmetic composition as defined above, for caring for and effacing the signs of age, such as wrinkles and fine lines on the skin, and for moisturizing the skin and/or the lips.

Moreover, the composition according to the invention is suitable for treating dry skin and it is also well tolerated by sensitive skin. Thus, another subject of the present invention is the cosmetic use of a cosmetic composition as defined above, for caring for, protecting and/or making up sensitive and/or dry skin.

The composition according to the invention is especially suitable for effacing the signs of ageing of the skin, and especially wrinkles and fine lines on the skin.

A subject of the present invention is also a cosmetic process for treating the signs of ageing of the skin, characterized in that a cosmetic composition as defined above is applied to the skin.

The examples below of compositions according to the invention are given as illustrations and with no limiting nature. The amounts therein are given as weight percentages, unless otherwise mentioned.

The procedure was the same for all the examples, and consisted in emulsifying by dispersing the oily phase in the aqueous phase at 75° C.

### EXAMPLES 1 AND 2 ACCORDING TO THE INVENTION AND COMPARATIVE EXAMPLE 3

| | | | |
|---|---|---|---|
| | Example 1 | Example 2 | |
| | according to | according to | Comparative |
| Composition | the invention | the invention | Example 3 |

Aqueous phase

| | | | |
|---|---|---|---|
| Crosslinked AMPS | 1.5 | 1.5 | — |
| polymer containing | | | |
| hydrophobic units | | | |
| (Aristoflex HMS) | | | |
| Crosslinked AMPS | — | — | 1.5 |
| homopolymer | | | |
| (Hostacerin AMPS) | | | |
| Glycerol | 5 | 5 | 5 |
| Preserving agent | 0.3 | 0.3 | 0.3 |
| Water | qs 100% | qs 100% | qs 100% |

Oily phase

| | | | |
|---|---|---|---|
| Apricot kernel oil | 10 | 10 | 10 |
| Parleam oil | 5 | 10 | 5 |
| Polystearyl | 1 | 1 | 1 |
| acrylate | | | |
| (Intelimer IPA 13-1 | | | |
| from Landec) | | | |
| Result after 2 | Stable | Stable | Unstable |
| months at 45° C. | | | |

Comparative Example 3 shows that using an AMPS polymer not containing hydrophobic units does not make it possible to obtain a stable composition with a semi-crystalline polymer, whereas, conversely, replacing the AMPS homopolymer with the hydrophobic AMPS copolymer makes it possible to stabilize a composition containing a semi-crystalline polymer.

Examples 1 and 2 according to the invention show that the combination of the crosslinked hydrophobic AMPS copolymer and of the semi-crystalline polymer in accordance with the invention makes it possible to obtain a stable composition even when the oil content is increased and large.

### EXAMPLE 4 ACCORDING TO THE INVENTION AND COMPARATIVE EXAMPLE 5

| | | |
|---|---|---|
| | Example 4 | |
| | according to | Comparative |
| Composition | the invention | example 5 |

Aqueous phase

| | | |
|---|---|---|
| Glycerol | 5% | 5% |
| NaOH | qs pH 7 | qs pH 7 |
| Xanthan gum | 0.2 | 0.2 |
| Crosslinked AMPS polymer | 0.3 | — |
| containing hydrophobic units | | |
| (Aristoflex HMS) | | |
| Non-crosslinked AMPS polymer | — | 0.3 |
| containing hydrophobic units | | |
| (Aristoflex LNC) | | |

Oily phase

| | | |
|---|---|---|
| Polystearyl acrylate | 1.5% | 1.5% |
| (Intelimer IPA 13-1 from | | |
| Landec) | | |
| Stearic acid | 1% | 1% |
| Beeswax | 1% | 1% |
| Palm oil and hydrogenated | 1% | 1% |
| palm kernel oil (Lipocire DM | | |
| from Gattefosse) | | |
| Cyclohexasiloxane | 3.5% | 3.5% |
| Sucrose mono-di-palmito- | 1% | 1% |
| stearate (Tegosoft PSE 141 G) | | |
| Apricot oil | 8% | 8% |
| Liquid fraction of shea | 2% | 2% |
| butter | | |
| Result after two weeks | Stable white | Unstable phase- |
| | emulsion | separated emulsion |

Comparative Example 5 shows that non-crosslinked AMPS polymers containing hydrophobic units do not make it possible to achieve the aim of the invention.

### EXAMPLE 5 ACCORDING TO THE INVENTION

### Smoothing Fluid

Oily phase

| | |
|---|---|
| Polystearyl acrylate (Intelimer IPA 13-1 | 1.5% |
| from Landec) | |
| Liquid fraction of shea butter | 4% |
| Cyclohexasiloxane | 3.5% |
| Beeswax | 0.7% |
| Apricot kernel oil | 8% |
| Cyclopentasiloxane | 4% |

Aqueous phase

| | |
|---|---|
| Preserving agent | 1% |
| Crosslinked copolymer of AMPS and of | 0.8% |
| 25 EO stearyl methacrylate (Aristoflex HMS) | |
| Glycerol | 5% |
| Sucrose mono-di-palmito-stearate | 1% |
| (Tegosoft PSE 141 G) | |
| Water | qs 100% |

A quite fluid cream was obtained (viscosity, measured using a Rheomat viscometer with a No. 3 spindle, of about 18 poises).

This cream may be used for smoothing out wrinkles.

Test: a test with this fluid cream was performed on a panel of 20 individuals. This test showed that 80% of the individuals found the cream to be soft and fresh, and that it had a smoothing, pouty and moisturizing effect on the skin, and also a soft-focus effect (effacing of skin defects).

### EXAMPLE 6 ACCORDING TO THE INVENTION

### Refreshing Repairing Melting Balm

Oily phase

| | |
|---|---|
| Polystearyl acrylate (Intelimer IPA 13-1 | 2% |
| from Landec) | |
| Liquid fraction of shea butter | 4% |
| Cyclohexasiloxane | 3.5% |
| Beeswax | 1% |
| Squalane | 6.5% |

Aqueous phase

| | |
|---|---|
| Preserving agent | 1% |
| Crosslinked copolymer of AMPS and of | 1% |
| 25 EO stearyl methacrylate (Aristoflex HMS) | |
| Glycerol | 5% |
| Water | qs 100% |

A quite thick cream was obtained (viscosity, measured using a Rheomat viscometer with a No. 5 spindle, of about 400 poises).

This cream may be used for smoothing out, moisturizing and nourishing the skin and for treating wrinkles and fine lines.

Test: a test with this cream was performed on 50 individuals. This test showed that the texture was judged to be firm and compact on taking up with the fingers, that the composition melted on application to the skin, giving a fresh sensation, and that it penetrated without leaving any tacky or greasy film. After application, the skin felt soft, fresh, matt and smooth, the wrinkles and fine lines on the skin having been effaced.

### COMPARATIVE EXAMPLES 7 AND 8

These examples are similar to Example 1, with replacement of the polystearyl acrylate (Intelimer IPA 13-1) with other polymers:

| | Example 1 | | |
|---|---|---|---|
| | according to | Comparative | Comparative |
| Composition | the invention | Example 7 | Example 8 |
| Crosslinked AMPS | 1.5 | 1.5 | 1.5 |
| polymer containing | | | |
| hydrophobic units | | | |
| (Aristoflex HMS) | | | |
| Acrylates/Beheneth- | | 1 | — |
| 25 Methacrylate | | | |
| Copolymer (Aculyn 28 | | | |
| from Rohm & Haas) | | | |
| Acrylates/C10-30 | — | — | 1 |
| Alkyl Acrylate | | | |
| Crosspolymer | | | |
| (Pemulen TR1 from | | | |
| Noveon) | | | |
| Glycerol | 5 | 5 | 5 |
| Preserving agent | 0.3 | 0.3 | 0.3 |
| Water | qs 100% | qs 100% | qs 100% |
| Apricot kernel oil | 10 | 10 | 10 |
| Parleam oil | 5 | 5 | 5 |
| Polystearyl acrylate | 1 | — | — |
| (Intelimer IPA | | | |
| 13-1 from Landec) | | | |
| Appearance | Creamy | Impossible to | Emulsion |
| | emulsion | make the | of brittle |
| | that melts | emulsion: the | texture that |
| | on the skin | two phases do | breaks into |
| | | not mix together | pieces on |
| | | and remain | attempting |
| | | impossible to | to spread it |
| | | homogenize | on the skin. |
| | | | Unpleasant |
| | | | appearance |

In this table, the amounts are indicated as weight of active material. The polymer Intelimer IPA-13-1 was replaced with an equivalent active material amount of Aculyn 28 (Comparative Example 7) or of Pemulen (Comparative Example 8).

1. A composition for topical application, comprising:
at least one crosslinked 2-acrylamido-2-methylpropanesulfonic acid polymer comprising at least one hydrophobic unit, and
at least one semi-crystalline polymer,
wherein the composition is in the form of an O/W emulsion comprising an oily phase dispersed in an aqueous phase.

2. The composition according to claim 1, wherein the crosslinked 2-acrylamido-2-methylpropanesulfonic acid polymer comprising at least one hydrophobic unit is partially or totally neutralized with a mineral or organic base.

3. The composition according to claim 1 wherein the crosslinked 2-acrylamido-2-methylpropanesulfonic acid polymer comprises:
from 80 mol % to 99 mol % of 2-acrylamido-2-methylpropanesulfonic acid (AMPS) units of formula (I): 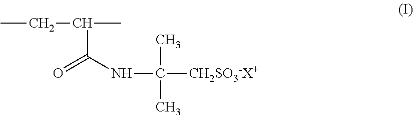
wherein
X⁺ is a proton, an alkali metal cation, an alkaline-earth metal cation, an ammonium ion or an organic cation; and
from 1 mol % to 20 mol % of hydrophobic units of formula (II): 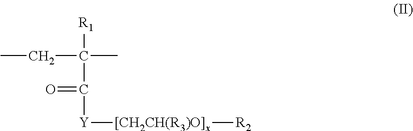
wherein
R₁ and R₃, which may be identical or different, is a hydrogen atom or a linear or branched C₁-C₆ alkyl radical;
Y is O or NH;
R₂ is a hydrocarbon-based radical containing from 6 to 50 carbon atoms; and
x is a number of moles of alkylene oxide and ranges from 0 to 100.

4. The composition according to claim 3, wherein the hydrophobic unit is a unit of formula (III): 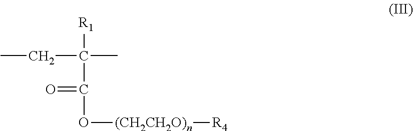
wherein
n is an integer ranging from 3 to 100;
R₁ is hydrogen or a methyl radical and
R₄ is a linear or branched alkyl radical comprising from 6 to 22 carbon atoms.

5. The composition according claim 1, wherein the crosslinked 2-acrylamido-2-methylpropanesulfonic acid polymer comprising at least one hydrophobic unit is obtained by polymerization of 2-acrylamido-2-methylpropanesulfonic acid or of a sodium or ammonium salt thereof with an ester of methacrylic acid and of an oxyethylenated alcohol of formula (IV):
—CH═C(CH₃)—CO—O(CH₂CH₂O)n-R (IV)
wherein n ranges from 7 to 25 and R is an alkyl residue comprising from 10 to 22 carbon atoms.

6. The composition according to claim 1, wherein the crosslinked 2-acrylamido-2-methylpropanesulfonic acid polymer comprising at least one hydrophobic unit is selected from the group consisting of AMPS/ethoxylated (25 EO) stearyl methacrylate crosslinked copolymer, AMPS/ethoxylated (25 EO) behenyl methacrylate crosslinked copolymer, and mixtures thereof.

7. The composition according to claim 1, wherein an amount of crosslinked 2-acrylamido-2-methylpropanesulfonic acid polymer(s) comprising at least one hydrophobic unit ranges from 0.1% to 5% by weight relative to the total weight of the composition.

8. The composition according to claim 1, wherein the semi-crystalline polymer comprises at least one of homopolymers and copolymers resulting from the polymerization of at least one monomer with (a) crystallizable chain(s) comprising at least 11 carbon atoms and not more than 40 carbon atoms.

9. The composition according to claim 8, wherein the semi-crystalline polymer is a homopolymer resulting from the polymerization of a monomer with (a) crystallizable chain(s) comprising saturated C₁₄-C₂₂ alkyl acrylates, saturated C₁₄-C₂₂ alkyl methacrylates or mixtures thereof.

10. The composition according to claim 1, wherein an amount of semi-crystalline polymer(s) ranges from 0.1% to 5% by weight relative to the total weight of the composition.

11. The composition according to claim 1, wherein the oily phase represents from 2% to 40% by weight relative to the total weight of the composition.

12. The composition according to claim 1, wherein the oily phase comprises at least one oil and at least one wax and/or one pasty fatty substance.

13. The composition according to claim 12, wherein an amount of wax(es) and of pasty fatty substance(s) ranges from 0.05% to 10% by weight relative to the total weight of the composition.

14. A method for caring for, protecting and/or making up the skin and/or the lips, comprising applying a cosmetic composition comprising the composition according to claim 1, to the skin and/or lips.

15. A method for moisturizing the skin and/or the lips, comprising applying a cosmetic composition comprising the composition according to claim 1, to the skin and/or lips.

16. A skin care composition for treating the signs of ageing of the skin, comprising the composition of claim 1 in a cosmetically acceptable medium.
